(19) United States
(12) Patent Application Publication
(10) Pub. No.: US 2018/0110757 A1
(43) Pub. Date: Apr. 26, 2018

(54) METHODS OF TREATING PRURITIS AND PAIN

(71) Applicant: The Regents of the University of California, Oakland, CA (US)

(72) Inventors: Diana M. Bautista, Berkeley, CA (US); Takeshi Morita, Albany, CA (US); Rose Zabel Hill, Berkeley, CA (US); Rachel B. Brem, Novato, CA (US)

(21) Appl. No.: 15/788,545

(22) Filed: Oct. 19, 2017

Related U.S. Application Data
(60) Provisional application No. 62/410,734, filed on Oct. 20, 2016.

(51) Int. Cl. A61K 31/426; A61K 31/4418; A61K 31/404; A61K 31/54; A61K 45/06

(52) U.S. Cl. CPC A61K 31/426; A61K 31/4418; A61K 45/06; A61K 31/54; A61K 31/404

(57) ABSTRACT

The present disclosure provides compositions and methods for treating pruritis and for treating certain types of pain.

### CROSS-REFERENCE

This application claims the benefit of U.S. Provisional Patent Application No. 62/410,734, filed Oct. 20, 2016, which application is incorporated herein by reference in its entirety.

### STATEMENT REGARDING FEDERALLY SPONSORED RESEARCH

This invention was made with government support under Grant Nos. AR059385 and N5077224 awarded by the National Institutes of Health. The government has certain rights in the invention.

### INTRODUCTION

Chronic itch is a debilitating condition that is highly prevalent; however, the molecular mechanisms of chronic itch are poorly understood. Current therapies to treat chronic itch broadly target the skin barrier (e.g., with topically applied creams) or the immune system (e.g., antihistamines; steroids; immunosuppressant drugs).

There is a need in the art for compositions and methods of treating chronic itch.

### SUMMARY

The present disclosure provides compositions and methods for treating pruritis. The present disclosure provides compositions and methods for treating pain.

### BRIEF DESCRIPTION OF THE DRAWINGS

FIG. 1A-1G depict the role of sphingosine-1-phosphate receptor type 3 (S1PR3) in pruritis.

### DEFINITIONS

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an S1PR3 antagonist” includes a plurality of such antagonists and reference to “the symptom” includes reference to one or more symptoms and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a method of treating itch in an individual, the method comprising administering to the individual an effective amount of an S1PR3 antagonist. The present disclosure provides a method of treating pain (e.g., mechanical pain; inflammatory pain) in an the method comprising administering to the individual an effective amount of an S1PR3 antagonist and/or an agent that reduces production of sphingosine-1-phosphate.

Methods of Treating Pruritis

The present disclosure provides a method of treating itch in an individual, the method comprising administering to the individual an effective amount of an S1PR3 antagonist.

In some cases, an “effective amount” of an S1PR3 antagonist is an amount that is effective to reduce itch (the sensation of itching) by at least 10%, at least 25%, at least 50%, at least 75%, at least 80%, at least 90%, or 100%, for a period of time of from about 1 hour to about 24 hours, from about 1 day to about 7 days, from about 1 week to about 4 weeks, from about 1 month to about 6 months, or more than 6 months. In some cases, an “effective amount” of an S1PR3 antagonist is an amount that is effective to reduce the urge to scratch by at least 10%, at least 25%, at least 50%, at least 75%, at least 80%, at least 90%, or 100%, for a period of time of from about 1 hour to about 24 hours, from about 1 day to about 7 days, from about 1 week to about 4 weeks, from about 1 month to about 6 months, or more than 6 months. In some cases, an “effective amount” of an S1PR3 antagonist is an amount that is effective to reduce outward manifestations of the cause of itch (e.g., hives, etc.) by at least 10%, at least 25%, at least 50%, at least 75%, at least 80%, at least 90%, or 100%, for a period of time of from about 1 hour to about 24 hours, from about 1 day to about 7 days, from about 1 week to about 4 weeks, from about 1 month to about 6 months, or more than 6 months. In some cases, an effective amount of an S1PR3 inhibitor is an amount that is effective to reduce the level of S1PR3 mRNA in a tissue of an individual (e.g., a skin tissue affected by itch) by at least 10%, at least 25%, at least 50%, at least 75%, at least 80%, at least 90%, or 100%, for a period of time of from about 1 hour to about 24 hours, from about 1 day to about 7 days, from about 1 week to about 4 weeks, from about 1 month to about 6 months, or more than 6 months.

In some cases, an effective amount of an S1PR3 inhibitor is an amount that is effective to reduce the level of S1PR3 mRNA in a tissue of an individual. In some cases, a method of the present disclosure comprises: a) administering to an individual in need thereof an effective amount of an S1PR3 inhibitor; and b) detecting the level of S1PR3 mRNA in a tissue of the individual (e.g., in a skin tissue that is affected by itch). In some cases, a method of the present disclosure comprises: a) administering to an individual in need thereof an effective amount of an S1PR3 inhibitor; b) detecting the level of S1PR3 mRNA in a tissue of the individual (e.g., in a skin tissue that is affected by itch); and c) based on the level detected in step (b), adjusting one or more of: i) the amount of the S1PR3 inhibitor administered; ii) the frequency of administration of the S1PR3 inhibitor; and the duration of administration of the S1PR3 inhibitor.

In some cases, S1PR3 inhibitor is selective for the S1PR3 receptor; e.g., the S1PR3 inhibitor inhibits the S1PR3 receptor subtype but does not substantially inhibit any other S1P receptor subtype. In some cases, the S1PR3 inhibitor inhibits an S1PR3 receptor, and one other S1PR subtype.

S1PR₃ Inhibitors

Sphingosine-1-phosphate (S1P), a member of the lysophospholipid family, is a signaling messenger and lipid mediator. S1P is produced by the sphingosine kinase-catalyzed phosphorylation of sphingosine. Signals initiated by S1P are transduced by the S1P receptors, which are a group of G-protein-coupled receptors that are divided into 5 subtypes: S1P₁, S1P₂, S1P₃, S1P₄, and S1P₅. These receptors mediate the bioactivity of S1P and are activated by binding to S1P. The term “S1PR3,” as used herein, refers to an S1PR3 receptor, and can be used interchangeably with S1P₃R, EDG3, or Edg-3 in the present disclosure.

The present disclosure provides herein a method of treating pruritis, where the method comprises administering to the individual an effective amount of an S1PR₃ inhibitor. Administration of an antagonist (i.e. inhibitor) of an S1PR₃ receptor to an individual in need thereof results in inhibition of the S1PR₃ receptor, and treatment of acute or chronic itch in the individual.

Suitable S1PR₃ inhibitors include those disclosed in U.S. patent application Ser. No. 11/675,168, No. 60/884,470, No. 11/690,637, and No. 60/824,807, No. 12/749,331, in International Patent Application No. WO 2003/062,392 and No. WO 2012/123,613, and in U.S. Pat. No. 8,563,594. The disclosures of all the foregoing references are incorporated herein by reference in their entirety.

In some cases, a suitable S1PR₃ inhibitor is a compound of Formula (I):

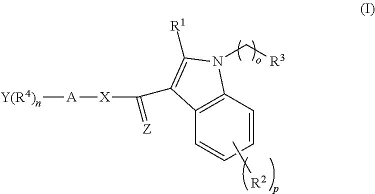

where:

X is NR⁵, O, or S;

Z is O or S;

n is O or an integer of from 1 to 4;

o is 0 or an integer of from 1 to 3;

p is 0 or an integer of from 1 to 4;

A is (C(R⁵)₂)ₘ, wherein

m is 0 or an integer of from 1 to 6;

R⁵ is selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, wherein said aryl is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, halo, C₁ to C₁₂ haloalkyl, hydroxyl, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl and sulfonyl groups;

Y is a carbocyclic aryl or heterocyclic aryl group; wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, and wherein said aryl may be bonded to A at any position;

R¹, R², R³, R⁴ are each independently selected from the group consisting of hydrogen; straight or branched chain alkyl having 1 to 12 carbons; cycloalkyl having 3 to 6 carbons; alkenyl having 2 to 6 carbons and 1 or 2 double bonds; alkynyl having 2 to 6 carbons and 1 or 2 triple bonds; aryl wherein said aryl is a carbocyclic aryl or heterocyclic aryl group, wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur; halo; C₁ to C₁₂ haloalkyl; hydroxyl; C₁ to C₁₂ alkoxy; C3 to C20 arylalkyloxy; C₁ to C₁₂ alkylcarbonyl; formyl; oxycarbonyl; carboxy; C₁ to C₁₂ alkyl carboxylate; C₁ to C₁₂ alkyl amide; aminocarbonyl; amino; cyano; diazo; nitro; thio; sulfoxyl; sulfonyl groups; or a group selected from the group consisting of

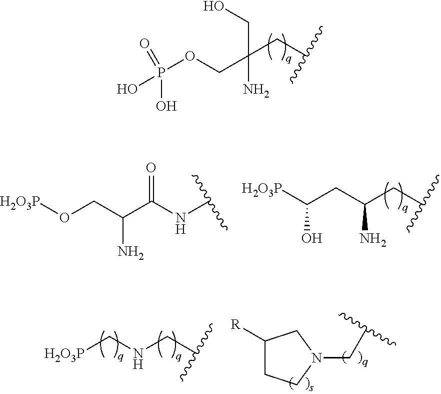

wherein R is CO₂H or PO₃H₂, p is an integer of 1 or 2 and q is 0 or an integer of 1 to 5 and s is 0 or an integer of 1 or 2; provided that, if Y is phenyl, it must be substituted with at least one R⁴ group that is not hydrogen.

In some cases, a suitable S1PR₃ inhibitor is a compound of Formula (ID:

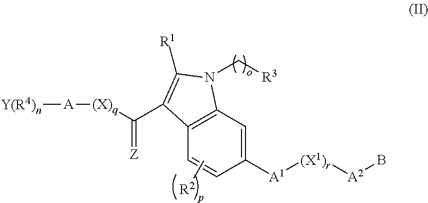

where:

R¹, R², R³ and R⁴ are independently selected from the group consisting of hydrogen, straight or branched chain alkyl having 1 to 12 carbons, alkenyl having 2 to 6 carbons and 1 or 2 double bonds, alkynyl having 2 to 6 carbons and 1 or 2 triple bonds, carbocyclic hydrocarbon groups having from 3 to 20 carbon atoms, heterocyclic groups having up to 20 carbon atoms and at least one of oxygen, nitrogen and/or sulfur in the ring, halo, C₁ to C₁₂ haloalkyl, hydroxyl, C₁ to C₁₂ alkoxy, C₃ to C₂₀ arylalkyloxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl, and sulfonyl groups;

X and X¹ are independently selected from the group consisting of NR⁵, O and S;

R⁵ is hydrogen, an alkyl group of 1 to 10 carbons, a cycloalkyl group of 5 to 10 carbons, phenyl or lower alkylphenyl;

Y is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, and wherein said aryl may be bonded to A at any position;

Z is O or S;

n is 0 or an integer of from 1 to 5;

o is 0 or an integer of from 1 to 3;

p is 0 or an integer of from 1 to 3;

q is 0 or 1;

r is 0 or 1;

A, A¹ and A² are independently selected from the group consisting of (CH₂)ᵥ wherein v is 0 or an integer of from 1 to12, branched chain alkyl having 3 to 12 carbons, cycloalkyl having 3 to 12 carbons, alkenyl having 2 to 10 carbons and 1-3 double bonds and alkynyl having 2 to 10 carbons and 1 to 3 triple bonds;

B is selected from the group consisting of hydrogen, OR⁶, COOR⁷, NR⁸R⁹, CONR⁸R⁹, COR¹⁰, CH═NOR¹¹, CH═NNR¹²R¹³ wherein R⁶, R⁷, R¹⁰ and R¹¹ are independently selected from the group consisting of hydrogen, straight or branched chain alkyl having 1 to 12 carbons, alkenyl having 2 to 6 carbons and 1 or 2 double bonds, alkynyl having 2 to 6 carbons and 1 or 2 triple bonds, a carbocyclic hydrocarbon group having from 3 to 20 carbon atoms, a heterocyclic group having up to 20 carbon atoms and at least one of oxygen, nitrogen and/or sulfur in the ring, R⁸, R⁹, R¹² and R¹³ are independently selected from the group consisting of hydrogen, straight or branched chain alkyl having 1 to 12 carbons, alkenyl having 2 to 6 carbons and 1 or 2 double bonds, alkynyl having 2 to 6 carbons and 1 or 2 triple bonds, a carbocyclic hydrocarbon group having from 3 to 20 carbon atoms, a heterocyclic group having up to 20 carbon atoms and at least one of oxygen, nitrogen and/or sulfur in the ring, or R⁸ and R⁹ and/or R¹² and R¹³, together, can form a divalent carbon radical of 2 to 5 carbons to form a heterocyclic ring with nitrogen, wherein any of R⁶, R⁷, R⁸, R⁹, R¹⁰, R¹¹, R¹² or R¹³ (may be substituted with one or more halogen, hydroxy,alkyloxy, cyano, nitro, mercapto or thiol radical; provided however, when v is 0, and r is 0, B is not hydrogen; or B is a carbocyclic hydrocarbon group having from 3 to 20 carbon atoms, or a heterocyclic group having up to 20 carbon atoms and at least one of oxygen, nitrogen and/or sulfur in the ring, and wherein when said B is a carbocyclic or heterocyclic group B may be bonded to A² at any position, or a pharmaceutically acceptable salt of said compound.

The aryl group is a carbocyclic aryl or heterocyclic aryl group wherein said carbocylic aryl comprises from 6 to 20 atoms and said heterocyclic aryl comprise from 2 to 20 carbon atoms and from 1 to 5 heteroatoms selected from the group consisting of nitrogen, oxygen and sulfur, and preferably said aryl group is selected from the group consisting of benzene, pyridine, pyrazine, pyridazine, pyrimidine, triazine, thiophene, furan, thiazole, thiadiazole, isothiazole, oxazole, oxadiazole, isooxazole, naphthalene, quinoline, tetralin, chroman, thiochroman, tetrahydroquinoline, dihydronaphthalene, tetrahydronaphthalen, chromene, thiochromene, dihydroquinoline, indan, dihydrobenzofuran, dihydroben, zothiophene, indene, benzofuran, benzothiophene, coumarin and coumarinone. The aryl groups can be bonded to the above moiety at any position. The aryl group may itself be substituted with any common organic functional group including but not limited to C₁ to C₁₂ alkyl, C₂ to C₆ alkenyl, 10 C₂ to C₆ alkynyl, halo, C₁ to C₁₂ haloalkyl, hydroxyl, C₁ to C₁₂ alkoxyl, C₁ to C₁₂ alkylcarbonyl, formyl, oxycarbonyl, carboxyl, C₁ to C₁₂ alkyl carboxylate, C₁ to C₁₂ alkyl amide, aminocarbonyl, amino, cyano, diazo, nitro, thio, sulfoxyl, or sulfonyl groups.

In some cases, Z is O.

In some cases, the carbocyclic aryl group will comprise from 6 to 14 carbon atoms, e.g. from 6 to 10 carbon atoms. In some cases, the heterocyclic aryl group will comprise from 2 to 14 carbon 5 atoms and one or more, e.g. from 1 to 3 heteroatoms selected from the group consisting of nitrogen, oxygen and sulfur.

In some cases, A is CH₂.

In some cases, X is NH.

In some cases, n is 0 or an integer of 1 or 2, and R⁴ is fluoro.

In some cases, R¹ is i-propyl;

In some cases, R³ is phenyl, which may be substituted with one or two fluoro groups; or pyridyl.

In some cases, p is 0.

In some cases, A¹ and A² are absent.

In some cases, B is OR⁶ or COOR⁷.

In some cases, X is O, r is 1, A¹ is absent, A² is (CH₂)ᵥ, wherein v is 1 or 2, and B is OR⁶ or NR⁸R⁹, and R⁶, R⁸ and R⁹ are methyl.

In some cases, B is CR¹⁰═NOR¹¹R¹⁰ wherein R¹⁰ is H and

R¹¹ is methyl or i-butyl or B is CONR⁸R⁹ wherein R⁸ and R⁹ are selected from the group consisting of H, methyl, ethyl and propyl, or R⁸ and R⁹, together with N, form a 5-member ring.

In some cases, A¹ is absent, r is 0, A² is CH₂ and B is OR⁶, wherein R⁶ is H, or X is O, r is 1 and B is COR¹⁰, wherein R¹⁰ is methyl.

In some cases, a suitable S1PR₃ inhibitor is a compound of Formula (III):

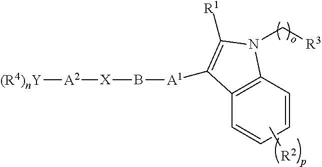

where:

A¹ and A² are independently selected from the group consisting of (CH₂)m where m is 0 or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, NR⁵, O and S;

B is selected from the group consisting of (CH₂)n, where n is 0 or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, C═C(R⁵) 2, C═O, C═S, R⁵C NR⁵, R⁵C═CR⁵, C═NOR⁵, CR⁵OR⁵, C(OR⁵)₂ , CR⁵N(R⁵)₂ , C(N(R⁵)₂)₂ , CR⁵SR⁵, C(SR⁵)₂ , SO, SO₂ , and heterocyclic aryl comprising from 2 to 14 carbon atoms and from 1 to 3 heteroatoms selected from the group consisting of nitrogen, oxygen and sulfur;

X is selected from the group consisting of (CH₂)r, where r is 0 or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, NR⁵, O and S; provided that when m is 0 and B is C═O then X is not NR⁵, O or S;

Y is R⁶, or a carbocyclic aryl group comprising from 6 to 14 carbon atoms or a heterocyclic aryl group comprising from 2 to 14 carbon atoms and from 1 to 3 heteroatoms selected from the group consisting of nitrogen, oxygen and sulfur;

o is 0 or an integer of from 1 to 3;

p is 0 or an integer of from 1 to 4;

R¹, R², R³, R⁴ are independently selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, halo, C₁ to C₁₂ haloalkyl, hydroxy, c1 to c12 alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl, sulfonyl,

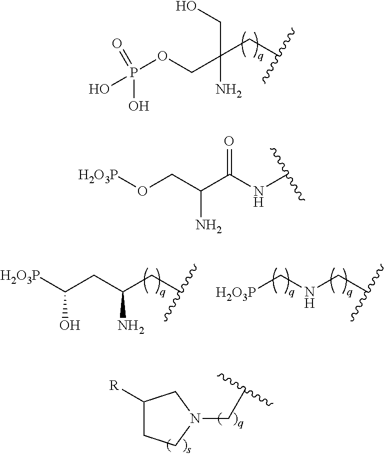

wherein R is CO₂H or PO₃H₂ and q is 0 or an integer of 1 to 5 and sis 0 or an integer from 1 to 3;

R⁵ is selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, halo, C₁ to C₁₂ haloalkyl, hydroxyl, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl and sulfonyl; and

R⁶ is selected from the group consisting of straight or branched chain alkyl having 1 to 12 carbons, cycloalkyl having 3 to 6 carbons, alkenyl having 2 to 6 carbons and 1 or 2 double bonds and alkynyl having 2 to 6 carbons and 1 or 2 triple bonds.

In some cases, a suitable S1PR₃ inhibitor is a compound of Formula (IV):

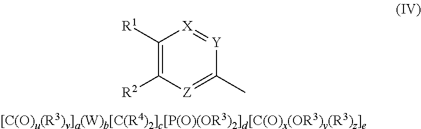

where:

X is selected from the group consisting of CR³ and N;

Y is selected from the group consisting of CR³ and N;

Z is selected from the group consisting of CR³ and N;

at least one of X, Y and Z is N;

W is NR³ or O;

R¹ is an aryl group;

R² is an aryl group;

R³ is selected from the group consisting of H and alkyl; and 2 of said R³ groups may together with N may form a heterocylic ring having from 2 to 6 carbon atoms; R⁴ is selected from the group consisting of H, alkyl, OR³, and N(R³)₂;

a is 0 or an integer of from 1 to 6;

b is 0 or 1;

c is 0 or an integer of from 1 to 6;

d is 0 or 1;

e is 0 or 1;

u is 0 or 1;

v is 0 or an integer of from 1 to 2;

xis 0 or 1;

y is 0 or an integer of from 1 to 3;

z is 0 or an integer of from 1 to 3; provided, however, that when d is 0, e is 1, and when e is 0, d is 1.

In some cases, a suitable S1PR₃ inhibitor (an Edg-3 inhibitor) includes a compound of the following structure [2-(2,4,6-Trimethyl-phenylamino)-benzo[1,3]-dioxol-2-yl]-carbamic acid ethyl ester:

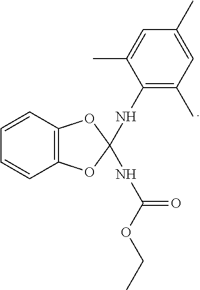 [SMILES: CCOC(=O)NC1(Nc2c(C)cc(C)cc2C)Oc2ccccc2O1]

In some cases, a suitable Edg-3 inhibitor includes a compound of the following structure [3-methyl-2-phenyl-quinoline-4 carboxylic acid 4-fluoro-benzylamide]:

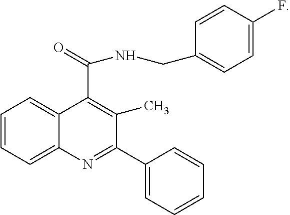 [SMILES: Cc1c(-c2ccccc2)nc2ccccc2c1C(=O)NCc1ccc(F)cc1]

See, e.g., WO2003/062392A2.

In some cases, a suitable S1PR₃ inhibitor is an antagonist of the S1PR₃ receptor, where the antagonist attenuates binding affinity or specificity between the S1PR₃ receptor and its natural ligand, S1P. The antagonist may be a S1P analog. Antagonists may be a substituted thiazolidine, e.g., an alkyl-substituted thiazolidine or an arylalkyl-substituted thiazolidine, a substituted thiazinane particularly an alkyl-substituted thiazinane, a polysulfonated naphthylurea such as suramin (most commonly available as the hexasodium salt), or a S1P analog, an antibody, biologically active antibody fragment thereof, peptide or a peptidomimetic having binding specificity and affinity for the S1PR₃ receptor; or a pharmaceutically acceptable salt of an antagonist. Antagonist agents as set forth herein may be a racemic mixture, a diastereomer or an enantiomer.

In the present disclosure, a “pharmaceutically acceptable salt of an antagonist” is a salt of an antagonist that retains the S1PR₃ receptor antagonistic activity and is acceptable by the human body. Salts may be acid or base salts.

In some cases, a suitable S1PR₃ antagonist is a compound of Formula (V):

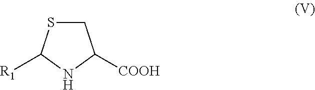

where:

R₁ is C₆-C₁₃ alkyl, or alkyl-substituted aryl where the substitution is C₅-C₉ alkyl. In one embodiment, the antagonist has structure of Formula V where R₁ is C₁₀ alkyl or C₁₁ alkyl, (2-alkylthiaZolidine-4-carboxylic acid, where the alkyl is C₁₀ or CH). In some cases, when R₁ is C₁₁ alkyl, the antagonist is CAY10444 available commercially from Cayman Chemical (Ann Arbor, Mich.). In other cases, the antagonist is a compound of formula V where R₁ is alkyl-substituted phenyl and the substitution on the phenyl ring is m- or p C₇-alkyl i.e., (2-(m- or p-heptylphenyl) thiazolidine-4-carboxylic acid).

In some cases, a suitable S1PR₃ antagonist is a compound of Formula (VI):

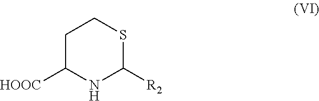

where R₂ is C₉-C₁₃ alkyl.

In some cases, a suitable S1PR₃ antagonist is a compound of Formula (VII):

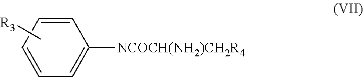

where:

R₃ is o- or m-C₅-C₈ alkyl; and R₄ is phosphate, phosphate analog, phosphonate, or sulfate. As used herein “phosphate analog” includes the terms phosphoro-thioates, -dithioates, -selenoates, -diselenoates, -anilothioates, -anilidates, -amidates, or boron phosphates, for example. See, e.g., U.S. Patent Publication No. 2010/183,629.

In some cases, a suitable S1PR₃ antagonist is a noncompetitive antagonist selective for

SiP₁ and S1PR₃ (K Takabe et al., 2008. Pharmacol Rev 60: 181-195, Dong-Soon I Sinica Acta Pharmacol 2010, 31: 1213-1222). In some cases, the S1PR₃ antagonist is VPC23019 ((R) phosphoric-acid mono-[2-amino-2-(3-octyl-phenylcarbamoyl)-ethyl]ester) compound with the following structure:

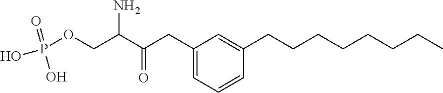 [SMILES: CCCCCCCCc1cccc(CC(=O)C(N)COP(=O)(O)O)c1]

In some cases, a suitable S1PR₃ antagonist is VPC25239 (Takabe K et al Pharmacol Rev 2008, 60: 181-195, Dong-Soon I Sinica Acta Pharmacol 2010, 31: 1213-1222). VPC25239 has the following structure:

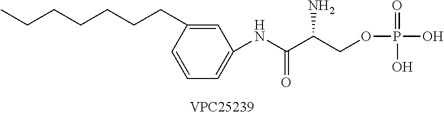 [SMILES: CCCCCCCc1cccc(NC(=O)[C@H](N)COP(=O)(O)O)c1]

In some cases, a suitable S1PR₃ antagonist is VPC01091 (((1R,3S)-1-amino-3-(3-octylphenyl)cyclopentyl)methanol; Dong-Soon I, Acta Pharmacol 2010, 31: 1213-1222). In some cases, a suitable S1PR3 antagonist is Suramin (8-[(4-methyl-3-{[3-({[3-({2-methyl-5-[(4,6,8-trisulfo-1-naphthyl)carbamoyl]phenyl}carbamoyl)phenyl]carbamoyl}amino) benzoyl]amino}benzoyl)amino]naphthalene-1,3,5-trisulfonic acid).

In some cases, a suitable S1PR₃ antagonist is BML-241 (2-undecyl-thiazolidine-4-ácidocarboxílico) compound, also referred to as CAY10444 of the following structure:

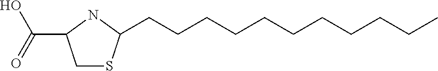 [SMILES: CCCCCCCCCCCC1NC(C(=O)O)CS1]

See, e.g., WO 2012/123,613.

In some cases, a suitable S1PR₃ inhibitor is an antibody. Antibodies having binding specificity and affinity for the S1PR₃ receptor are available commercially, for example, a mouse monoclonal antibody is available from GENETEX, Inc. (Catalog Number GTX12254, San Antonio, Tex.); and the EDG-3 CT antibody is available from Exalpha Biologicals, Inc. (Watertown, Mass). EDG-3 CT has binding affinity and specificity for the unique C-terminal peptide of human S1P₃ receptor. See, e.g., U.S. Patent Publication No. 2010/183,629. In some cases, the antibody is a humanized antibody.

Further compounds active in S1PR₃ signaling are described in US. Patent Application Publication No. 2005/0222422 to Lynch et al., published Oct. 6, 2005, incorporated by reference herein, and Koide et al., J Med Chem, Vol.45:4629-4638, 2002.

Formulations, Dosages, Routes of Administration

As discussed above, a treatment method of the present disclosure generally involves administering to an individual in need thereof an effective amount of an S1PR3 antagonist. For simplicity, the term “agent” or “active agent”, below, refers to an S1PR3 antagonist. Formulations, dosages, and routes of administration are discussed below. In some cases, a composition, e.g., a pharmaceutical composition, comprising an active compound is administered to an individual in need thereof.

In some instances, a composition comprising an active agent (an S1PR3 antagonist) can comprise a pharmaceutically acceptable excipient, a variety of which are known in the art and need not be discussed in detail herein. Pharmaceutically acceptable excipients have been amply described in a variety of publications, including, for example, A. Gennaro (1995) “Remington: The Science and Practice of Pharmacy”, 19th edition, Lippincott, Williams, & Wilkins.

For simplicity, in the following discussion of formulations, dosages, and routes of administration, an S1PR3 antagonist as described above is referred to as an “agent,” “drug,” or “active agent.”

Formulations

In the subject methods, the active agent(s) may be administered to the host using any convenient means capable of resulting in the desired therapeutic effect or clinical outcome. Thus, an active agent can be incorporated into a variety of formulations for therapeutic administration. More particularly, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, an active agent may be administered in the form of its pharmaceutically acceptable salt, or an active agent may also be used alone or in appropriate association, as well as in combination, with other pharmaceutically active compounds. The following methods and excipients are merely exemplary and are in no way limiting.

For oral preparations, an active agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An active agent can be utilized in aerosol formulation to be administered via inhalation.

An active agent can be formulated into pressurized acceptable propellants such as dichlorodifluoromethane, propane, nitrogen and the like.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise an active agent in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of an active agent calculated in an amount sufficient to produce the desired effect in association with a pharmaceutically acceptable diluent, carrier or vehicle. The specifications for a suitable dosage form depend, e.g., on the particular active agent employed and the effect to be achieved, and the pharmacodynamics associated with each compound in the host.

Other modes of administration will also find use with a subject treatment method. For instance, an active agent can be formulated in suppositories and, in some cases, aerosol and intranasal compositions. For suppositories, the vehicle composition can include traditional binders and carriers such as, polyalkylene glycols, or triglycerides. Such suppositories may be formed from mixtures containing the active ingredient in the range of about 0.5% to about 10% (w/w), e.g., about 1% to about 2%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of an active agent by the nasal mucosa.

An active agent can be administered in a composition suitable for injection. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Oral Formulations

In some embodiments, an active agent is formulated for oral delivery to an individual in need of such an agent.

For oral delivery, a formulation comprising an active agent will in some embodiments include an enteric-soluble coating material. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit™, and shellac.

As one non-limiting example of a suitable oral formulation, an active agent is formulated with one or more pharmaceutical excipients and coated with an enteric coating, as described in U.S. Pat. No. 6,346,269. For example, a solution comprising an active agent and a stabilizer is coated onto a core comprising pharmaceutically acceptable excipients, to form an active agent-coated core; a sub-coating layer is applied to the active agent-coated core, which is then coated with an enteric coating layer. The core generally includes pharmaceutically inactive components such as lactose, a starch, mannitol, sodium carboxymethyl cellulose, sodium starch glycolate, sodium chloride, potassium chloride, pigments, salts of alginic acid, talc, titanium dioxide, stearic acid, stearate, micro-crystalline cellulose, glycerin, polyethylene glycol, triethyl citrate, tributyl citrate, propanyl triacetate, dibasic calcium phosphate, tribasic sodium phosphate, calcium sulfate, cyclodextrin, and castor oil. Suitable solvents for an active agent include aqueous solvents. Suitable stabilizers include alkali-metals and alkaline earth metals, bases of phosphates and organic acid salts and organic amines The sub-coating layer comprises one or more of an adhesive, a plasticizer, and an anti-tackiness agent. Suitable anti-tackiness agents include talc, stearic acid, stearate, sodium stearyl fumarate, glyceryl behenate, kaolin and aerosil. Suitable adhesives include polyvinyl pyrrolidone (PVP), gelatin, hydroxyethyl cellulose (HEC), hydroxypropyl cellulose (HPC), hydroxypropyl methyl cellulose (HPMC), vinyl acetate (VA), polyvinyl alcohol (PVA), methyl cellulose (MC), ethyl cellulose (EC), hydroxypropyl methyl cellulose phthalate (HPMCP), cellulose acetate phthalates (CAP), xanthan gum, alginic acid, salts of alginic acid, Eudragit™, copolymer of methyl acrylic acid/methyl methacrylate with polyvinyl acetate phthalate (PVAP). Suitable plasticizers include glycerin, polyethylene glycol, triethyl citrate, tributyl citrate, propanyl triacetate and castor oil. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit™ and shellac.

Suitable oral formulations also include an active agent formulated with any of the following: microgranules (see, e.g., U.S. Pat. No. 6,458,398); biodegradable macromers (see, e.g., U.S. Pat. No. 6,703,037); biodegradable hydrogels (see, e.g., Graham and McNeill (1989) Biomaterials 5:27-36); biodegradable particulate vectors (see, e.g., U.S. Pat. No. 5,736,371); bioabsorbable lactone polymers (see, e.g., U.S. Pat. No. 5,631,015); slow release protein polymers (see, e.g., U.S. Pat. No. 6,699,504; Pelias Technologies, Inc.); a poly(lactide-co-glycolide/polyethylene glycol block copolymer (see, e.g., U.S. Pat. No. 6,630,155; Atrix Laboratories, Inc.); a composition comprising a biocompatible polymer and particles of metal cation-stabilized agent dispersed within the polymer (see, e.g., U.S. Pat. No. 6,379,701; p Alkermes Controlled Therapeutics, Inc.); and microspheres (see, e.g., U.S. Pat. No. 6,303,148; Octoplus, B. V.).

Suitable oral formulations also include an active agent formulated with any of the following: a carrier such as Emisphere® (Emisphere Technologies, Inc.); TIMERx, a hydrophilic matrix combining xanthan and locust bean gums which, in the presence of dextrose, form a strong binder gel in water (Penwest); Geminex™ (Penwest); Procise™ (GlaxoSmithKline); SAVIT™ (Mistral Pharma Inc.); RingCap™ (Alza Corp.); Smartrix® (Smartrix Technologies, Inc.); SQZgel™ (MacroMed, Inc.); Geomatrix™ (Skye Pharma, Inc.); Oros® Tri-layer (Alza Corporation); and the like.

Also suitable for use are formulations such as those described in U.S. Pat. No. 6,296,842 (Alkermes Controlled Therapeutics, Inc.); U.S. Pat. No. 6,187,330 (Scios, Inc.); and the like.

Also suitable for use herein are formulations comprising an intestinal absorption enhancing agent. Suitable intestinal absorption enhancers include, but are not limited to, calcium chelators (e.g., citrate, ethylenediamine tetracetic acid); surfactants (e.g., sodium dodecyl sulfate, bile salts, palmitoylcarnitine, and sodium salts of fatty acids); toxins (e.g., zonula occludens toxin); and the like.

Controlled Release Formulations

In some embodiments, an active agent is formulated in a controlled release formulation.

Controlled release within the scope of this disclosure can be taken to mean any one of a number of extended release dosage forms. The following terms may be considered to be substantially equivalent to controlled release, for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.).

The various controlled release technologies cover a very broad spectrum of drug dosage forms. Controlled release technologies include, but are not limited to physical systems and chemical systems.

Physical systems include, but are not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., nonerodible, erodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous). Additional discussion of categories of systems for controlled release may be found in Agis F. Kydonieus, Controlled Release Technologies: Methods, Theory and Applications, 1980 (CRC Press, Inc.).

There are a number of controlled release drug formulations that are developed for oral administration. These include, but are not limited to, osmotic pressure-controlled gastrointestinal delivery systems; hydrodynamic pressure-controlled gastrointestinal delivery systems; membrane permeation-controlled gastrointestinal delivery systems, which include microporous membrane permeation-controlled gastrointestinal delivery devices; gastric fluid-resistant intestine targeted controlled-release gastrointestinal delivery devices; gel diffusion-controlled gastrointestinal delivery systems; and ion-exchange-controlled gastrointestinal delivery systems, which include cationic and anionic drugs. Additional information regarding controlled release drug delivery systems may be found in Yie W. Chien, Novel Drug Delivery Systems, 1992 (Marcel Dekker, Inc.). Some of these formulations will now be discussed in more detail.

Enteric coatings are applied to tablets to prevent the release of drugs in the stomach either to reduce the risk of unpleasant side effects or to maintain the stability of the drug which might otherwise be subject to degradation of expose to the gastric environment. Most polymers that are used for this purpose are polyacids that function by virtue or the fact that their solubility in aqueous medium is pH-dependent, and they require conditions with a pH higher than normally encountered in the stomach.

One exemplary type of oral controlled release structure is enteric coating of a solid or liquid dosage form. The enteric coatings are designed to disintegrate in intestinal fluid for ready absorption. Delay of absorption of the active agent that is incorporated into a formulation with an enteric coating is dependent on the rate of transfer through the gastrointestinal tract, and so the rate of gastric emptying is an important factor. In one exemplary embodiment, an active agent can be contained in an enterically coated multiple-unit dosage form. In an exemplary embodiment, a dosage form comprising an active agent is prepared by spray-coating granules of the active agent-enteric coating agent solid dispersion on an inert core material. These granules can result in prolonged absorption of the active agent with good bioavailability.

Typical enteric coating agents include, but are not limited to, hydroxypropylmethylcellulose phthalate, methacryclic acid-methacrylic acid ester copolymer, polyvinyl acetate-phthalate and cellulose acetate phthalate. Akihiko Hasegawa, Application of solid dispersions of Nifedipine with enteric coating agent to prepare a sustained-release dosage form, Chem. Pharm. Bull. 33: 1615-1619 (1985). Various enteric coating materials may be selected on the basis of testing to achieve an enteric coated dosage form designed ab initio to have an optimal combination of dissolution time, coating thicknesses and diametral crushing strength. S. C. Porter et al., The Properties of Enteric Tablet Coatings Made From Polyvinyl Acetate-phthalate and Cellulose acetate Phthalate, J. Pharm. Pharmacol. 22:42 p (1970).

Another type of useful oral controlled release structure is a solid dispersion. A solid dispersion may be defined as a dispersion of one or more active ingredients in an inert carrier or matrix in the solid state prepared by the melting (fusion), solvent, or melting-solvent method. Akihiko Hasegawa, Super Saturation Mechanism of Drugs from Solid Dispersions with Enteric Coating Agents, Chem. Pharm. Bull. 36: 4941-4950 (1998). The solid dispersions may be also called solid-state dispersions. The term “coprecipitates” may also be used to refer to those preparations obtained by the solvent methods.

The selection of the carrier may have an influence on the dissolution characteristics of the dispersed active agent because the dissolution rate of a component from a surface may be affected by other components in a multiple component mixture. For example, a water-soluble carrier may result in a fast release of the drug from the matrix, or a poorly soluble or insoluble carrier may lead to a slower release of the drug from the matrix. The solubility of an active agent may also be increased owing to some interaction with the carriers.

Examples of carriers useful in solid dispersions include, but are not limited to, water-soluble polymers such as polyethylene glycol, polyvinylpyrrolidone, and hydroxypropylmethyl-cellulose. Alternative carriers include phosphatidylcholine. Phosphatidylcholine is an amphoteric but water-insoluble lipid, which may improve the solubility of otherwise insoluble active agents in an amorphous state in phosphatidylcholine solid dispersions.

Other carriers include polyoxyethylene hydrogenated castor oil. Poorly water-soluble active agents may be included in a solid dispersion system with an enteric polymer such as hydroxypropylmethylcellulose phthalate and carboxymethylethylcellulose, and a non-enteric polymer, hydroxypropylmethylcellulose. Another solid dispersion dosage form includes incorporation of an active agent with ethyl cellulose and stearic acid in different ratios.

There are various methods commonly known for preparing solid dispersions. These include, but are not limited to, the melting method, the solvent method and the melting-solvent method.

Another controlled release dosage form is a complex between an ion exchange resin and an active agent. Ion exchange resin-drug complexes have been used to formulate sustained-release products of acidic and basic drugs. In one exemplary embodiment, a polymeric film coating is provided to the ion exchange resin-drug complex particles, making drug release from these particles diffusion controlled. See Y. Raghunathan et al., Sustained-released drug delivery system I: Coded ion-exchange resin systems for phenylpropanolamine and other drugs, J. Pharm. Sciences 70: 379-384 (1981).

Injectable microspheres are another controlled release dosage form. Injectable micro spheres may be prepared by non-aqueous phase separation techniques, and spray-drying techniques. Microspheres may be prepared using polylactic acid or copoly(lactic/glycolic acid). Shigeyuki Takada, Utilization of an Amorphous Form of a Water-Soluble GPIIb/IIIa Antagonist for Controlled Release From Biodegradable Micro spheres, Pharm. Res. 14:1146-1150 (1997), and ethyl cellulose, Yoshiyuki Koida, Studies on Dissolution Mechanism of Drugs from Ethyl Cellulose Microcapsules, Chem. Pharm. Bull. 35:1538-1545 (1987).

Other controlled release technologies that may be used include, but are not limited to, SODAS (Spheroidal Oral Drug Absorption System), INDAS (Insoluble Drug Absorption System), IPDAS (Intestinal Protective Drug Absorption System), MODAS (Multiporous Oral Drug Absorption System), EFVAS (Effervescent Drug Absorption System), PRODAS (Programmable Oral Drug Absorption System), and DUREDAS (Dual Release Drug Absorption System) available from Elan Pharmaceutical Technologies. SODAS are multi particulate dosage forms utilizing controlled release beads. INDAS are a family of drug delivery technologies designed to increase the solubility of poorly soluble drugs. IPDAS are multi particulate tablet formation utilizing a combination of high density controlled release beads and an immediate-release granulate. MODAS are controlled release single unit dosage forms. Each tablet consists of an inner core surrounded by a semipermeable multiparous membrane that controls the rate of drug release. EFVAS is an effervescent drug absorption system. PRODAS is a family of multi particulate formulations utilizing combinations of immediate release and controlled release mini-tablets. DUREDAS is a bilayer tablet formulation providing dual release rates within the one dosage form. Although these dosage forms are known to one of skill, certain of these dosage forms will now be discussed in more detail.

INDAS was developed specifically to improve the solubility and absorption characteristics of poorly water soluble drugs. Solubility and, in particular, dissolution within the fluids of the gastrointestinal tract is a key factor in determining the overall oral bioavailability of poorly water soluble drug. By enhancing solubility, one can increase the overall bioavailability of a drug with resulting reductions in dosage. INDAS takes the form of a high energy matrix tablet, production of which is comprised of two distinct steps: the drug in question is converted to an amorphous form through a combination of energy, excipients, and unique processing procedures.

Once converted to the desirable physical form, the resultant high energy complex may be stabilized by an absorption process that utilizes a novel polymer cross-linked technology to prevent recrystallization. The combination of the change in the physical state of an active agent coupled with the solubilizing characteristics of the excipients employed enhances the solubility of the active agent. The resulting absorbed amorphous drug complex granulate may be formulated with a gel-forming erodible tablet system to promote substantially smooth and continuous absorption.

IPDAS is a multi-particulate tablet technology that may enhance the gastrointestinal tolerability of potential irritant and ulcerogenic drugs. Intestinal protection is facilitated by the multi-particulate nature of the IPDAS formulation which promotes dispersion of an irritant lipoate throughout the gastrointestinal tract. Controlled release characteristics of the individual beads may avoid high concentration of drug being both released locally and absorbed systemically. The combination of both approaches serves to minimize the potential harm of an active agent with resultant benefits to patients.

IPDAS is composed of numerous high density controlled release beads. Each bead may be manufactured by a two step process that involves the initial production of a micromatrix with embedded active agent and the subsequent coating of this micromatrix with polymer solutions that form a rate-limiting semipermeable membrane in vivo. Once an IPDAS tablet is ingested, it may disintegrate and liberate the beads in the stomach. These beads may subsequently pass into the duodenum and along the gastrointestinal tract, e.g., in a controlled and gradual manner, independent of the feeding state. Release of the active agent occurs by diffusion process through the micromatrix and subsequently through the pores in the rate controlling semipermeable membrane. The release rate from the IPDAS tablet may be customized to deliver a drug-specific absorption profile associated with optimized clinical benefit. Should a fast onset of activity be necessary, immediate release granulate may be included in the tablet. The tablet may be broken prior to administration, without substantially compromising drug release, if a reduced dose is required for individual titration.

MODAS is a drug delivery system that may be used to control the absorption of water soluble agents. Physically MODAS is a non-disintegrating table formulation that manipulates drug release by a process of rate limiting diffusion by a semipermeable membrane formed in vivo. The diffusion process essentially dictates the rate of presentation of drug to the gastrointestinal fluids, such that the uptake into the body is controlled. Because of the minimal use of excipients, MODAS can readily accommodate small dosage size forms. Each MODAS tablet begins as a core containing active drug plus excipients. This core is coated with a solution of insoluble polymers and soluble excipients. Once the tablet is ingested, the fluid of the gastrointestinal tract may dissolve the soluble excipients in the outer coating leaving substantially the insoluble polymer. What results is a network of tiny, narrow channels connecting fluid from the gastrointestinal tract to the inner drug core of water soluble drug. This fluid passes through these channels, into the core, dissolving the drug, and the resultant solution of drug may diffuse out in a controlled manner This may permit both controlled dissolution and absorption. An advantage of this system is that the drug releasing pores of the tablet are distributed over substantially the entire surface of the tablet. This facilitates uniform drug absorption reduces aggressive unidirectional drug delivery. MODAS represents a very flexible dosage form in that both the inner core and the outer semipermeable membrane may be altered to suit the individual delivery requirements of a drug. In particular, the addition of excipients to the inner core may help to produce a microenvironment within the tablet that facilitates more predictable release and absorption rates. The addition of an immediate release outer coating may allow for development of combination products.

Additionally, PRODAS may be used to deliver an active agent. PRODAS is a multi particulate drug delivery technology based on the production of controlled release mini tablets in the size range of 1.5 to 4 mm in diameter. The PRODAS technology is a hybrid of multi particulate and hydrophilic matrix tablet approaches, and may incorporate, in one dosage form, the benefits of both these drug delivery systems.

In its most basic form, PRODAS involves the direct compression of an immediate release granulate to produce individual mini tablets that contain an active agent. These mini tablets are subsequently incorporated into hard gels and capsules that represent the final dosage form. A more beneficial use of this technology is in the production of controlled release formulations. In this case, the incorporation of various polymer combinations within the granulate may delay the release rate of drugs from each of the individual mini tablets. These mini tablets may subsequently be coated with controlled release polymer solutions to provide additional delayed release properties. The additional coating may be necessary in the case of highly water soluble drugs or drugs that are perhaps gastroirritants where release can be delayed until the formulation reaches more distal regions of the gastrointestinal tract. One value of PRODAS technology lies in the inherent flexibility to formulation whereby combinations of mini tablets, each with different release rates, are incorporated into one dosage form. As well as potentially permitting controlled absorption over a specific period, this also may permit targeted delivery of drug to specific sites of absorption throughout the gastrointestinal tract. Combination products also may be possible using mini tablets formulated with different active ingredients.

DUREDAS is a bilayer tableting technology that may be used to an active agent. DUREDAS was developed to provide for two different release rates, or dual release of a drug from one dosage form. The term bilayer refers to two separate direct compression events that take place during the tableting process. In an exemplary embodiment, an immediate release granulate is first compressed, being followed by the addition of a controlled release element which is then compressed onto this initial tablet. This may give rise to the characteristic bilayer seen in the final dosage form.

The controlled release properties may be provided by a combination of hydrophilic polymers. In certain cases, a rapid release of an active agent may be desirable in order to facilitate a fast onset of therapeutic affect. Hence one layer of the tablet may be formulated as an immediate release granulate. By contrast, the second layer of the tablet may release the drug in a controlled manner, e.g., through the use of hydrophilic polymers. This controlled release may result from a combination of diffusion and erosion through the hydrophilic polymer matrix.

A further extension of DUREDAS technology is the production of controlled release combination dosage forms. In this instance, two different active agents may be incorporated into the bilayer tablet and the release of drug from each layer controlled to maximize therapeutic affect of the combination.

An active agent can be incorporated into any one of the aforementioned controlled released dosage forms, or other conventional dosage forms. The amount of active agent contained in each dose can be adjusted, to meet the needs of the individual patient, and the indication. One of skill in the art and reading this disclosure will readily recognize how to adjust the level of an active agent and the release rates in a controlled release formulation, in order to optimize delivery of the active agent and its bioavailability.

Inhalational Formulations

An active agent will in some embodiments be administered to a patient by means of a pharmaceutical delivery system for the inhalation route. An active agent may be formulated in a form suitable for administration by inhalation. The inhalational route of administration provides the advantage that the inhaled drug can bypass the blood-brain barrier. The pharmaceutical delivery system is one that is suitable for respiratory therapy by delivery of an active agent to mucosal linings of the bronchi. This invention can utilize a system that depends on the power of a compressed gas to expel an active agent from a container. An aerosol or pressurized package can be employed for this purpose.

As used herein, the term “aerosol” is used in its conventional sense as referring to very fine liquid or solid particles carries by a propellant gas under pressure to a site of therapeutic application. When a pharmaceutical aerosol is employed in this invention, the aerosol contains an active agent, which can be dissolved, suspended, or emulsified in a mixture of a fluid carrier and a propellant. The aerosol can be in the form of a solution, suspension, emulsion, powder, or semi-solid preparation. Aerosols employed in the present invention are intended for administration as fine, solid particles or as liquid mists via the respiratory tract of a patient. Various types of propellants known to one of skill in the art can be utilized. Suitable propellants include, but are not limited to, hydrocarbons or other suitable gas. In the case of the pressurized aerosol, the dosage unit may be determined by providing a value to deliver a metered amount.

An active agent can also be formulated for delivery with a nebulizer, which is an instrument that generates very fine liquid particles of substantially uniform size in a gas. For example, a liquid containing an active agent is dispersed as droplets. The small droplets can be carried by a current of air through an outlet tube of the nebulizer. The resulting mist penetrates into the respiratory tract of the patient.

A powder composition containing an active agent, with or without a lubricant, carrier, or propellant, can be administered to a mammal in need of therapy. This embodiment of the invention can be carried out with a conventional device for administering a powder pharmaceutical composition by inhalation. For example, a powder mixture of the compound and a suitable powder base such as lactose or starch may be presented in unit dosage form in for example capsular or cartridges, e.g. gelatin, or blister packs, from which the powder may be administered with the aid of an inhaler.

There are several different types of inhalation methodologies which can be employed in connection with a method of the present disclosure. An active agent can be formulated in basically three different types of formulations for inhalation. First, an active agent can be formulated with low boiling point propellants. Such formulations are generally administered by conventional meter dose inhalers (MDI's). However, conventional MDI's can be modified so as to increase the ability to obtain repeatable dosing by utilizing technology which measures the inspiratory volume and flow rate of the patient as discussed within U.S. Pat. Nos. 5,404,871 and 5,542,410.

Alternatively, an active agent can be formulated in aqueous or ethanolic solutions and delivered by conventional nebulizers. Lastly, an active agent can be formulated into dry powder formulations. Such formulations can be administered by simply inhaling the dry powder formulation after creating an aerosol mist of the powder.

Dosages

Although the dosage used will vary depending on the clinical goals to be achieved, a suitable dosage range is one which provides up to about 1 μg to about 1,000 μg or about 10,000 μg of an active agent and can be administered in a single dose. Alternatively, a target dosage of an active agent can be considered to be about in the range of about 0.1-1000 μM, about 0.5-500 μM, about 1-100 μM, or about 5-50 μM in a sample of host blood drawn within the first 24-48 hours after administration of the agent.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

Routes of Administration

An active agent is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, intracranial, subcutaneous, intradermal, topical application, intravenous, rectal, nasal, oral and other enteral and parenteral routes of administration. Routes of administration may be combined, if desired, or adjusted depending upon the agent and/or the desired effect. The composition can be administered in a single dose or in multiple doses. In some cases, the composition is administered orally. In other cases, the composition is administered intravenously. In other cases, the composition is administered via an inhalational route. In other cases, the composition is administered intramuscularly. In other cases, the composition is administered topically to the skin. In other cases, the composition is administered intradermally In other cases, the composition is administered subcutaneously. In other cases, the composition is administered transdermally.

The agent can be administered to a host using any available conventional methods and routes suitable for delivery of conventional drugs, including systemic or localized routes. In general, routes of administration contemplated by the present disclosure include, but are not necessarily limited to, enteral, parenteral, or inhalational routes. In some cases, an active agent is administered orally. In some cases, an active agent is administered topically. In some cases, an active agent is administered intradermally In some cases, an active agent is administered subcutaneously.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The agent can also be delivered to the subject by enteral administration. Enteral routes of administration include, but are not necessarily limited to, oral and rectal (e.g., using a suppository) delivery.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as a neurological disorder and pain that may be associated therewith. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts (wherein the term “host” is used interchangeably herein with the terms “individual,” “subject,” and “patient”) are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), humans, and non-human primates (e.g., chimpanzees, and monkeys). In some cases, the individual being treated will be a human.

Dosing

The formulation of an active agent and its subsequent administration (dosing) is within the skill of those in the art. Dosing can be dependent on one or more of several criteria, including severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual active agents, and can generally be estimated based on EC50s found to be effective in vitro and in vivo animal models.

For example, a suitable dose of an active agent is from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein an active agent is administered in maintenance doses, ranging from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight.

In some embodiments, multiple doses of an active agent are administered. The frequency of administration of an active agent can vary depending on any of a variety of factors, e.g., severity of the symptoms, etc. For example, in some embodiments, an active agent is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of an active agent, e.g., the period of time over which an active agent is administered, can vary, depending on any of a variety of factors, e.g., patient response, etc. For example, an active agent can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Combination Therapy

A subject method of treating itch can involve administering an active agent (an S1PR3 antagonist), and can further involve administering at least a second therapeutic agent. Suitable second therapeutic agents include, e.g., anti-inflammatory agents; topical or oral corticosteroids (e.g., hydrocortisone; betamethasone; fluticasone); a calcineurin inhibitor (e.g., pimecrolimus; tacrolimus); an antihistamine (e.g., diphenhydramine; hydroxyzine; etc.); cyclosporine; and an interferon.

Subjects Suitable for Treatment

Subjects suitable for treatment with a subject method include individuals having acute itch and individuals having chronic itch. Subjects suitable for treatment with a subject method include individuals having itch due to an allergic reaction. Subjects suitable for treatment with a subject method include individuals having itch due to contact with urushiol. Subjects suitable for treatment with a subject method include individuals having itch due to the presence on the individual of lice. Subjects suitable for treatment with a subject method include individuals having itch due to contact with poison ivy. Subjects suitable for treatment with a subject method include individuals having itch due to a Candida fungus infection of the skin. Subjects suitable for treatment with a subject method include individuals having itch due to tinea corporis. Subjects suitable for treatment with a subject method include individuals having itch due to contact with poison oak. Subjects suitable for treatment with a subject method include individuals having itch due to impetigo. Subjects suitable for treatment with a subject method include individuals having itch due to cutaneous larva migrans. Subjects suitable for treatment with a subject method include individuals having itch due to a herpes simplex virus infection. Subjects suitable for treatment with a subject method include individuals having itch due to an insect bite. Subjects suitable for treatment with a subject method include individuals having itch due to an arachnid bit. Subjects suitable for treatment with a subject method include individuals having itch due to photodermatitis. Subjects suitable for treatment with a subject method include individuals having itch due to scabies. Subjects suitable for treatment with a subject method include individuals having itch due to sarcoidosis. Subjects suitable for treatment with a subject method include individuals having itch due to anaphylaxis. Subjects suitable for treatment with a subject method include individuals having itch due to urticaria. Subjects suitable for treatment with a subject method include individuals having itch due to dandruff. Subjects suitable for treatment with a subject method include individuals having itch due to punctate palmoplantar keratoderma. Subjects suitable for treatment with a subject method include individuals having itch due to psoriasis. Subjects suitable for treatment with a subject method include individuals having itch due to eczema. Subjects suitable for treatment with a subject method include individuals having itch due to sunburn. Subjects suitable for treatment with a subject method include individuals having itch due to athlete's foot (fungal infection). Subjects suitable for treatment with a subject method include individuals having itch due to hidradenitis suppurativa. Subjects suitable for treatment with a subject method include individuals having itch due to xerosis. Subjects suitable for treatment with a subject method include individuals having itch due to uremic pruritis. Subjects suitable for treatment with a subject method include individuals having itch due to polycythemia. Subjects suitable for treatment with a subject method include individuals having itch due to dermatitis. Subjects suitable for treatment with a subject method include individuals having itch due to atopic dermatitis.

In some cases, the chronic itch being treated is secondary to a disease (other than a specific skin disease) such as a neurologic disorder, chronic renal failure, cholestasis, a systemic infection, an endocrine disorder, etc. Thus, in some cases, a subject suitable for treatment with a subject method is an individual having pruritis that is secondary to a disease other than a specific skin disease.

Methods for Detecting Pruritis

The present disclosure provides methods for detecting pruritis in an individual, the method comprising detecting levels of S1PR3 mRNA in a biological sample obtained from the individual. The methods can be used to determine the severity of pruritis. The methods can be used to assess whether an individual is responding to a treatment for pruritis.

Biological samples include tissue biopsies, e.g., skin biopsies, and the like. The biological sample can be treated in any of a variety of ways, e.g., following obtaining the sample from an individual. For example, a biological sample can be treated to enrich for mRNA.

In some cases, the level of S1PR3 mRNA in a biological sample is determined using a polymerase chain reaction (PCR), e.g., using primers specific for a cDNA copy of S1PR3 mRNA. The PCR can be quantitative PCR (qPCR). For example, in some cases, quantitative reverse transcription-polymerase chain reaction (qRT-PCR) is carried out, in which cDNA is generated, using as a template the mRNA in a biological sample; and qPCR is carried out on the cDNA, using primers specific for S1PR3. Where the level of S1PR3 mRNA is elevated, relative to a control S1PR3 level, a diagnosis of pruritis is indicated. Following a treatment for pruritis, where the level of S1PR3 mRNA is reduced, relative to a pre-treatment level of S1PR3 mRNA, the treatment can be considered efficacious for treating pruritis.

Methods of Treating Pain

The present disclosure provides methods of treating pain, e.g., mechanical pain and/or inflammatory pain. The methods generally involve administering to an individual in need thereof an effective amount of an S1PR3 antagonist and/or an effective amount of an agent that reduces production of sphingosine-1-phosphate (S1P). In some cases, the agent that reduces production of S1P is a sphingosine kinase inhibitor (SKI).

Subjects suitable for treatment include individuals having pain, e.g., an individual having neuropathic pain, central pain, deafferentiation pain, chronic pain, post-operative pain, pre-operative pain, nociceptive pain, acute pain, non-inflammatory pain, inflammatory pain, pain associated with cancer, wound pain, burn pain, pain associated with medical procedures, pain resulting from pruritus, painful bladder syndrome, pain associated with premenstrual dysphoric disorder, pain associated with premenstrual syndrome, pain associated with chronic fatigue syndrome, pain associated with pre-term labor, pain associated with drawal symptoms from drug addiction, joint pain, arthritic pain, lumbosacral pain, musculo-skeletal pain, headache, migraine, muscle ache, lower back pain, neck pain, toothache dental/maxillofacial pain, or visceral pain. Subjects suitable for treatment include individuals having pain, e.g., an individual who has experienced mechanical pain. Subjects suitable for treatment include individuals having pain, e.g., an individual having inflammatory pain. Individuals having inflammatory pain include individuals having inflammatory pain due to arthritis (e.g., rheumatoid arthritis; psoriatic arthritis; osteoarthritis; and the like). In some cases, the individual is one who does not have cancer, e.g., an individual who has not been diagnosed as having cancer.

In some cases, the individual has inflammatory pain due to an inflammatory condition. The term “inflammatory condition” refers to those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation.

Exemplary inflammatory conditions include, but are not limited to, inflammation associated with acne, anemia (e.g., aplastic anemia, hemolytic autoimmune anemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis, and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, type 2 diabetes mellitus), a skin condition (e.g., psoriasis, eczema, burns, dermatitis, pruritus (itch)), endometriosis, Guillain-Barre syndrome, infection, ischemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischemic colitis, diversion colitis, Behcet's syndrome, indeterminate colitis) and inflammatory bowel syndrome (IBS)), lupus, multiple sclerosis, morphea, myasthenia gravis, myocardial ischemia, nephrotic syndrome, pemphigus vulgaris, pernicious anemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, scleroderma, sarcoidosis, spondyloarthropathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatitis. In some cases, the inflammatory condition is an acute inflammatory condition (e.g., for example, inflammation resulting from infection). In some cases, the inflammatory condition is a chronic inflammatory condition (e.g., conditions resulting from asthma, arthritis and inflammatory bowel disease).

In some cases, an effective amount of an S1PR3 antagonist is an amount that reduces mechanical pain by at least 5%, at least 10%, at least 15%, at least 20%, at least 25%, at least 30%, at least 35%, at least 40%, at least 45%, at least 50%, at least 60%, at least 70%, at least 80%, or more than 80%, compared to the level of pain without treatment with the S1PR3 antagonist, or before treatment with the S1PR3 antagonist.

In some cases, an effective amount of an S1PR3 antagonist is an amount that reduces inflammatory pain by at least 5%, at least 10%, at least 15%, at least 20%, at least 25%, at least 30%, at least 35%, at least 40%, at least 45%, at least 50%, at least 60%, at least 70%, at least 80%, or more than 80%, compared to the level of pain without treatment with the S1PR3 antagonist, or before treatment with the S1PR3 antagonist.

In some cases, an effective amount of an SKI is an amount that reduces mechanical pain by at least 5%, at least 10%, at least 15%, at least 20%, at least 25%, at least 30%, at least 35%, at least 40%, at least 45%, at least 50%, at least 60%, at least 70%, at least 80%, or more than 80%, compared to the level of pain without treatment with the SKI, or before treatment with the SKI.

In some cases, an effective amount of an SKI is an amount that reduces inflammatory pain by at least 5%, at least 10%, at least 15%, at least 20%, at least 25%, at least 30%, at least 35%, at least 40%, at least 45%, at least 50%, at least 60%, at least 70%, at least 80%, or more than 80%, compared to the level of pain without treatment with the SKI, or before treatment with the SKI.

Suitable S1PR3 antagonists are described above.

Suitable sphingosine kinase inhibitors include, but are not limited to, SKI inhibitor is 2-(p-hydroxyanilino)-4-(p-chlorophenyl)thiazole (also known as SKI-II); (2R,3S,4E)-N-methyl-5-(4′-pentylphenyl)-2-aminopent-4-ene-1,3-diol (also known as BML-258); (3-(4-chlorophenyl)-adamantane-1-carboxylic acid (pyridin-4-ylmethyl)amide (ABC294640); (2-(p-Hydroxyanilino)-4-(p-chlorophenyl)thiazole); DMS (D-erythro-N,N-dimethylsphingosine); D,L-threo-dihydrosphingosine; N,N,N-trimethylsphingosine; (2R,3S,4E)-N-methyl-5-(4′-pentylphenyl)-2-aminopent-4-ene-1,3-diol; SKI-I (5-naphthalen-2-yl-2H-pyrazole-3-carboxylic acid (2-hydroxy-naphthalen-1-ylmethylene)-hydrazide); SKI-V (2-(3,4-Dihydroxy-benzylidene)-benzofuran-3-one); 5C (2,2-dimethyl-45-(1-oxo-2-hexadecyn-1-yl)-1,1-dimethylethyl ester-3-oxazolidinecarboxylic acid); ABC294640; ABC294735; and ABC747080. Suitable sphingosine kinase inhibitors are disclosed in US Patent Publication No. 2012/0095004.

ABC294735 has the following structure:

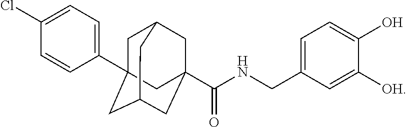 [SMILES: O=C(NCc1ccc(O)c(O)c1)C12CC3CC(C1)CC(c1ccc(Cl)cc1)(C3)C2]

ABC747080 and ABC294640 have the following structures.

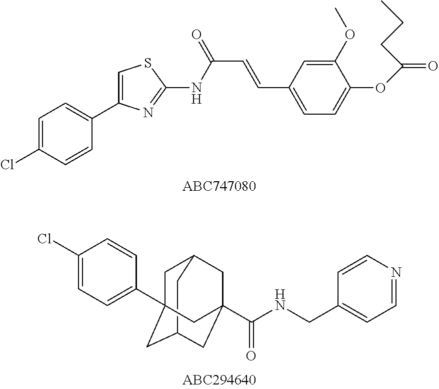 [SMILES: CCCC(=O)Oc1ccc(/C=C/C(=O)Nc2nc(-c3ccc(Cl)cc3)cs2)cc1OC ; O=C(NCc1ccncc1)C12CC3CC(C1)CC(c1ccc(Cl)cc1)(C3)C2]

SKI-II has the following structure:

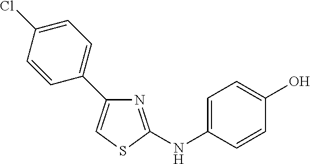 [SMILES: Oc1ccc(Nc2nc(-c3ccc(Cl)cc3)cs2)cc1]

Suitable formulations, routes of administration, and dosing are described above for S1PR3 antagonists, and apply to sphingosine kinase inhibitors.

Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-18 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A method of treating acute or chronic itch in an individual, the method comprising administering to the individual an effective amount of an S1PR3 inhibitor.

Aspect 2. The method of aspect 1, wherein the itch is chronic itch.

Aspect 3. The method of aspect 1, wherein the itch is due to psoriasis, eczema, or atopic dermatitis.

Aspect 4. The method of any one of aspects 1-3, wherein said administering is via topical administration.

Aspect 5. The method of any one of aspects 1-3, wherein said administering is via oral administration.

Aspect 6. The method of any one of aspects 1-3, wherein said administering is via intradermal administration.

Aspect 7. The method of any one of aspects 1-3, wherein said administering is via subcutaneous administration.

Aspect 8. The method of any one of aspects 1-7, wherein the individual is a human

Aspect 9. The method of any one of aspects 1-8, wherein the S1PR3 inhibitor is a compound of Formula I, as described above.

Aspect 10. The method of any one of aspects 1-8, wherein the S1PR3 inhibitor is a compound of Formula II, as described above.

Aspect 11. The method of any one of aspects 1-8, wherein the S1PR3 inhibitor is a compound of Formula III, as described above.

Aspect 12. The method of any one of aspects 1-8, wherein the S1PR3 inhibitor is a compound of Formula IV, as described above.

Aspect 13. The method of any one of aspects 1-8, wherein the S1PR3 inhibitor is a compound of Formula V, as described above.

Aspect 14. The method of any one of aspects 1-8, wherein the S1PR3 inhibitor is a compound of Formula VI, as described above.

Aspect 15. The method of any one of aspects 1-8, wherein the S1PR3 inhibitor is a compound of Formula VII, as described above.

Aspect 16. The method of any one of aspects 1-15, comprising administering a second therapeutic agent selected from an anti-inflammatory agents, a topical or oral corticosteroid, a calcineurin inhibitor, an antihistamine, cyclosporine, and an interferon.

Aspect 17. The method of any one of aspects 1-15, wherein the level of S1PR3 mRNA in a skin tissue of the individual is reduced.

Aspect 18. The method of any one of aspects 1-17, wherein the S1PR3 inhibitor is a selective S1PR3 inhibitor.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

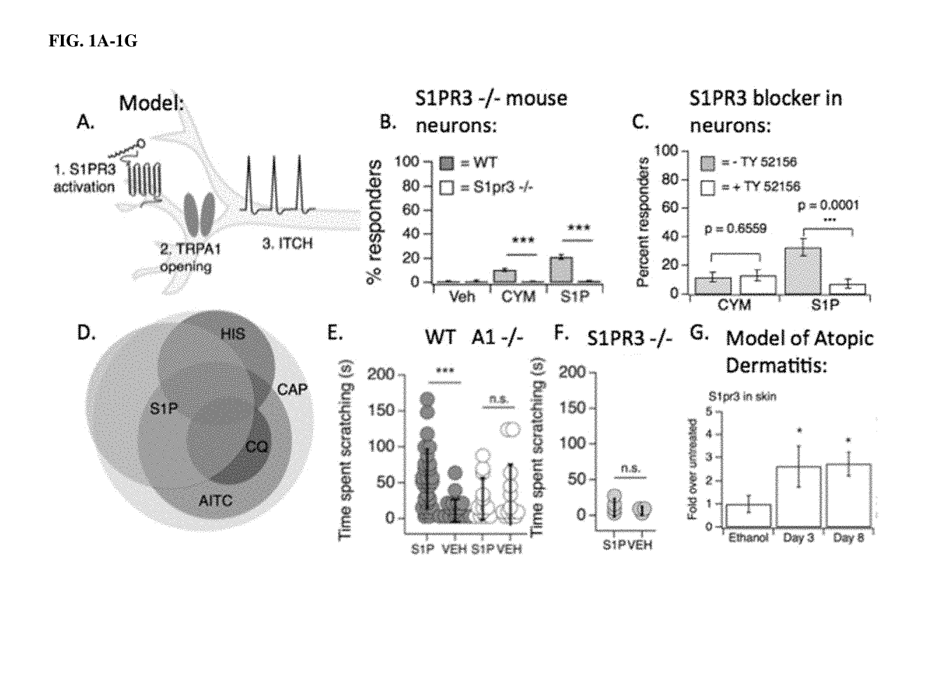

The data provided herein have revealed the mechanism by which S1PR3 evokes itch (FIG. 1A). Briefly, S1P can activate S1PR3, which then causes TRPA1 (an ion channel required for many forms of itch) opening in a subset of itch-sensing neurons. This S1PR3-dependent opening of TRPA1 then leads to scratching behavior in mice (FIG. 1E). Applying an S1PR3 inhibitor to sensory neurons prevents neurons from responding to S1P, and additionally S1PR3 is required for S1P-evoked itch (FIG. 1C and 1B). S1PR3 blockers may prevent endogenous S1P from causing itch, which may occur in allergic, atopic, and neuropathic conditions where S1P is elevated and/or dysregulated. Additionally, S1PR3 expression and activity can be assayed as a biomarker for chronic itch. The data indicate that S1PR3 mRNA transcripts are elevated in the skin of mice in a model of atopic dermatitis (eczema) (FIG. 1G). Thus S1PR3 levels can be assayed from skin biopsies from living patients as a readout and diagnostic for specific itch diseases.

FIG. 1A-1G. A. Graphical model illustrating the role of S1PR3-evoked itch in somatosensory neurons of the dorsal root ganglion (DRG). B. Ratiometric calcium imaging experiments demonstrate that S1PR3-deficient (S1PR3 −/−) mice lack calcium responses in cultured DRG neurons in response to addition of S1P (100 nM) or the S1PR3-specific agonist CYM 5541 (CYM, 100 μM), whereas wildtype mice (WT) display robust responses to both CYM and S1P compared to physiological saline control. C. Calcium imaging experiments demonstrate that the S1PR3-specific antagonist TY 52156 (N-(4-Chlorophenyl)-3,3-dimethyl-2-oxobutanimidic 2-(4-chlorophenyl) hydrazide) attenuates S1P-evoked calcium responses in wild-type DRG sensory neurons, but does not block the effects of the allosteric agonist CYM. D. Venn diagram displaying overlap of S1P responsive neurons from ratiometric calcium imaging with known pruritogens and algogens in cultured DRG neurons. Pruritogens: HIS=histamine, CQ=chloroquine. Algogens: CAP=capsaicin, AITC=allyl-isothiocyanate. Data were collected from n>4,000 neurons. E. Scratching behavioral data for mice injected with 200 nM S1P or 0.1% methanol-PBS vehicle intradermally into the rostral back. WT=wild-type, A1 −/−=Trpa1-deficient knockout mice. Scratching behaviors were recorded 30 minutes post injection. F. S1PR3-deficient mice (S1PR3 −/−) demonstrate no difference in scratching behavior to 200 nM S1P vs. vehicle. G. S1pr3 transcript levels were quantified using qRT-PCR from skin of mice treated with vitamin D or ethanol control for 7 days to induce an atopic dermatitis-like condition. Data were normalized to Gapdh housekeeping gene. For all data, one way ANOVA was performed with a Tukey-Kramer post hoc statistical test. Tukey-Kramer values are reported and only p<0.05 were considered significant, ***=p<0.001. n.s.=not significant.

### Example 2

A variety of somatosensory neurons mediate responses to diverse mechanical stimuli, from innocuous touch to noxious pain. While the transducers of light touch have been identified, the molecular mechanisms underlying mechanical pain remain largely unknown. Furthermore, due to this dearth of mechanistic knowledge and lack of effective therapies, mechanical pain remains the number one pain complaint in the clinic and can be debilitating for patients. Here it is shown that the bioactive lipid sphingosine 1-phosphate (S1P) and the S1P Receptor 3 (S1PR3) are critical regulators of acute mechanical pain and pain hypersensitivity. Genetic or pharmacological ablation of S1PR3, or blockade of S1P production, significantly impairs the detection of noxious mechanical stimuli, with no effect on innocuous touch or baseline thermal sensation. It was found that S1PR3 inhibition promoted analgesia in an inflammatory pain model. Without being bound to theory, it is proposed that the behavioral effects of S1PR3 signaling are mediated via tonic modulation of KCNQ channels to tune mechanonociceptor excitability. The findings establish the importance of S1P/S1PR3 signaling in the setting of mechanical pain thresholds and inflammatory pain hypersensitivity. S1PR3 antagonists and S1P synthesis inhibitors can be used for the treatment of mechanical and inflammatory pain.

S1PR3 is Required for Normal Mechanical Pain Sensation

S1pr3^{-/-} mice display a dramatic loss of mechanical pain sensitivity, as von Frey paw withdrawal thresholds were significantly elevated in S1pr3^{-/-} mice relative to S1pr3^{+/+} and S1pr3^{+/-} littermates. S1pr3^{-/-} mice also demonstrate decreased responsiveness to a range of noxious tactile stimuli (2-6 g), but normal responsiveness to innocuous tactile stimuli (0.6-1.4g). S1pr3^{-/-} mice exhibited normal light touch and proprioceptive responses, assayed by tape-removal attempts and righting reflexes. Likewise, in S1pr3^{-/-} mice, radiant heat withdrawal latencies were normal. These results demonstrate a selective role for S1PR3 in baseline mechanical pain.

Manipulation of S1P Levels or S1PR3 Signaling in the Periphery Heightens Mechanical Pain Thresholds While Sparing Gentle Touch Sensation in Healthy Mice

Pharmacological manipulation of S1PR3 or reduction of S1P levels lessens mechanical pain sensation in wild-type mice without affecting innocuous touch. Intradermal administration of the S1PR3-selective antagonist TY 52156 (TY) promotes mechanical hyposensitivity, and decreases responsiveness to noxious tactile stimuli, but not innocuous tactile stimuli.

Blockade of S1PR1 with the selective antagonist W146 has no effect on mechanosensitivity. Tonic levels of S1P in the skin are responsible for S1PR3-mediated mechanical pain sensation. Using an experimental paradigm in which injection of 50 μM sphingosine kinase inhibitor SKI II blocks local production of S1P, we found that mechanical pain sensitivity was dependent on local S1P in the skin.

The analgesic effects of acute S1PR3 antagonism or blocking S1P production were comparable to the hyposensitivity phenotype observed in S1pr3^{-/-} mice. These findings support a key role for peripheral S1PR3 signaling in setting mechanical pain thresholds and highlight the potential of S1PR3 blockers and S1P synthesis inhibitors for use as general analgesics that selectively target pain over other sensory modalities.

S1P/S1PR3 Signaling Closes KCNQ2/3 Channels to Increase Mechanical Nociceptor Excitability In Vitro

In situ RNA hybridization studies and protein localization studies indicate that S1pr3 is expressed in thermal nociceptors and mechanical nociceptors, as well as nociceptive free nerve endings in skin. S1PR3 increases the excitability of mechanical nociceptor DRG neurons in the presence of S1P through closure of KCNQ2/3 potassium channels (M channels).

Retigabine and other KCNQ modulators have shown promise as analgesics, although their crucial role in the excitability of central nervous system neurons makes them less useful as general analgesics. S1PR3 is less widely expressed than KCNQ2/3 channels in the central nervous system and therefore drugs targeting S1PR3 may present fewer side effects.

Blocking the S1P/S1PR3 Axis Alleviates Thermal and Mechanical Pain in the Complete Freund's Adjuvant (CFA) Model of Chronic Inflammation

S1PR3 knockout mice treated with CFA fail to develop thermal hypersensitivity and display normal mechanical sensitivity due to their baseline defect in mechanical pain sensation. They also display lessened paw swelling, possibly due to decreased neurogenic inflammation. Acute S1PR3 blockade with TY 52156 (delivered to the hindpaw after induction of CFA pain) elevates thermal and mechanical thresholds of wild-type mice to pre-CFA, baseline levels. Loss of S1P production with acute SKI II injection into the CFA-treated paw also reverses CFA thermal hypersensitivity.

Blood plasma is rich in S1P. Plasma extravasation in the CFA model may elevate local S1P concentrations leading to thermal hypersensitivity via S1PR3-mediated TRPV1 sensitization. S1P may also be an important inflammatory mediator in other diseases involving swelling due to plasma leakage. While many inflammatory molecules have been identified, S1PR3 is the first peripheral target that completely blocks the development of thermal hypersensitivity in the CFA model of chronic inflammatory pain, one of the most widely used animal models of pain. Taken together, the observations described herein suggest analgesia is mediated by simultaneous inhibition of the thermal nociceptors and mechanical nociceptors. These findings highlight the potential of selective S1PR3 antagonists and/or S1P blocking agents such as sphingosine kinase inhibitors for the treatment of mechanical and inflammatory pain in both healthy and sensitized individuals.

Additionally, a number of S1PR3 antagonists and S1P synthesis inhibitors have been tested for blocking chronic pruritis and thermal pain, including TY 52156 and SKI II. These compounds have great potential for pain and itch therapies.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of treating acute or chronic itch in an individual, the method comprising administering to the individual an effective amount of an S1PR3 inhibitor.

2. The method of claim 1, wherein the itch is chronic itch.

3. The method of claim 1, wherein the itch is due to psoriasis, eczema, or atopic dermatitis.

4. The method of claim 1, wherein said administering is via topical administration, via oral administration, via intradermal administration, or via subcutaneous administration.

5.-7. (canceled)

8. The method of claim 1, wherein the individual is a human.

9. The method of claim 1, wherein the S1PR3 inhibitor is a compound of Formula I, Formula II, Formula III, Formula IV, Formula V, Formula VI, or Formula VI.

10.-15. (canceled)

16. The method of claim 1, comprising administering a second therapeutic agent selected from an anti-inflammatory agents, a topical or oral corticosteroid, a calcineurin inhibitor, an antihistamine, cyclosporine, and an interferon.

17. The method of claim 1, wherein the level of S1PR3 mRNA in a skin tissue of the individual is reduced.

18. The method of claim 1, wherein the S1PR3 inhibitor is a selective S1PR3 inhibitor.

19. A method of reducing mechanical or inflammatory pain in an individual, the method comprising administering to the individual an effective amount of an S1PR3 inhibitor or an agent that reduces production of sphingosine-1-phosphate (S1P).

20. The method of claim 19, wherein said administering is via topical administration, via oral administration, via intradermal administration, or via intramuscular administration.

21.-23. (canceled)

24. The method of claim 19, wherein the individual is a human.

25. The method of claim 19, wherein the S1PR3 inhibitor is a compound of Formula I, Formula II, Formula III, Formula IV, Formula V, Formula VI, or Formula VI.

26.-31. (canceled)

32. The method of claim 19, wherein the S1PR3 inhibitor is a selective S1PR3 inhibitor.

33. The method of claim 19, wherein the agent that reduces production of S1P is a sphingosine kinase inhibitor (SKI).

34. The method of claim 33, wherein the SKI inhibitor is 2-(p-hydroxyanilino)-4-(p-chlorophenyl)thiazole.

35. The method of claim 33, wherein the SKI inhibitor is (2R,3S,4E)-N-methyl-5-(4′-pentylphenyl)-2-aminopent-4-ene-1,3-diol.

36. The method of claim 33, wherein the SKI inhibitor is (3-(4-chlorophenyl)-adamantane-1-carboxylic acid (pyridin-4-ylmethyl)amide.

37. (canceled)

38. The method of claim 33, wherein the SKI inhibitor is administered via oral, intramuscular, intravenous, or subcutaneous administration.
